(19) United States
(12) Patent Application Publication
(10) Pub. No.: US 2022/0288282 A1
(43) Pub. Date: Sep. 15, 2022

(54) INTERMITTENT CATHETERS

(71) Applicant: ConvaTec Limited, Flintshire (GB)

(72) Inventors: Daniel ALLEN, Flintshire (GB); David POLLARD, Flintshire (GB)

(21) Appl. No.: 17/693,586

(22) Filed: Mar. 14, 2022

Related U.S. Application Data
(63) Continuation of application No. PCT/GB2022/050646, filed on Mar. 14, 2022.

(30) Foreign Application Priority Data
Mar. 15, 2021 (GB) 2103549.8

(51) Int. Cl. A61L 29/16

(52) U.S. Cl. CPC A61L 29/16; A61L 2300/452; A61L 2300/216; A61L 2300/404

(57) ABSTRACT

The invention relates to intermittent catheters comprising a calixarene bonded to a surface thereof, in particular to provide a bacteria-repellent and/or lubricous coating on the outer surface of an intermittent catheter. The calixarene moiety may comprises hydrophilic surface groups to provide lubricity, whilst also providing a bacteria-repellent effect, especially useful preventing discomfort and/or infection for a user inserting multiple catheters per day.

### CROSS REFERENCE TO RELATED APPLICATIONS

The present disclosure is a continuation of International Application No. PCT/GB2022/050646 filed on Mar. 14, 2022 and claims the benefit of GB2103549.8 filed on Mar. 15, 2021, the contents of which are hereby incorporated herein by reference in entirety.

### TECHNICAL FIELD OF THE INVENTION

The present invention relates to intermittent catheters comprising a surface-coated calixarene that provides lubricity and/or bacteria-repellent properties, and to processes for manufacturing intermittent catheters.

### BACKGROUND TO THE INVENTION

Intermittent urinary catheterisation is a process involving insertion of a urinary catheter through an individual's urethra and into their bladder, where it is kept to empty the bladder of urine for only the time period that is required for emptying, after which the catheter is removed. The process differs from long-term catheterisation, which makes use of an indwelling or Foley catheter that is inserted into the bladder for long periods of time (several days to months) to discharge the residual urine of the bladder continuously throughout the day.

Intermittent catheterisation is often used by patients suffering from abnormalities of the urinary system, resulting in urinary incontinence and/or a lack of control in permitting voluntary urination. Such individuals would typically make use of intermittent catheters several times a day.

Intermittent catheters are useful devices, providing users with independence and freedom to self-catheterise as and when required, without having to rely on trained personnel to be present. This, however, increases the need for intermittent catheters to be user friendly: in particular, both easy to insert and remove with minimum discomfort caused, and safe to use with features for minimising risk of infection. Users often report experiencing pain and discomfort upon insertion and/or removal of intermittent catheters. Users have, for instance, reported experiencing bladder spasms, burning sensations, and bleeding. Urinary tract infections (UTI) are also common in individuals who practice intermittent catheterisation.

Coatings for intermittent catheters have been used to help alleviate some of these issues. However, patients have been found to develop sensitisation to such coatings, rendering their use less than ideal; particularly with intermittent catheters which a user would likely insert and remove multiple times a day.

United Kingdom Patent GB 2 448 153 describes implantable medical devices, including implantable vascular catheters, that contain a calixarene-based surface coating that is both hydrophobic and oleophobic.

United Kingdom Patent GB 2 498 356 also refers to implantable medical devices, including indwelling/Foley urinary catheters, that contain a calixarene-derived coating for resisting adhesion and/or colonisation of bacteria.

There exists a need for intermittent catheters that are both easier and less painful to insert and remove; and that are safer to use, with features for minimising risk of infection, for non-medically trained individuals practicing self-catheterisation.

It is an aim of embodiments of the present invention to address these requirements by providing an intermittent catheter, suitable for self-catheterisation use, which provides one or more of the following advantages:
A high lubricity, non-stick coating making the intermittent catheter easier to insert and remove, without the need for additional lubricants to be applied upon use.
An inert, bacteria repellent coating reducing the risk of infections developing (including UTIs) and ensuring that the intermittent catheter remains safe even when accidentally contacted by non-sterile objects.
A coating material to which a user has a decreased risk of developing sensitisation, even with such intermittent catheters being inserted and removed by the user multiple times a day.

It is also an aim of embodiments of the invention to overcome or mitigate at least one problem of the prior art, whether expressly described herein or not.

### SUMMARY OF THE INVENTION

According to one aspect of the invention, there is provided an intermittent catheter comprising a surface comprising a calixarene.

The intermittent catheter may comprise more than one calixarene on the surface, such as two, three or four.

Such a calixarene on an intermittent catheter provides high lubricity and bacteria repellent properties, making it both easier and safer to use, especially for individuals practicing self-catheterisation. Additionally, the user would have a decreased risk of developing sensitisation to the coating material, even with the user inserting and removing said intermittent catheters multiple times a day.

In some embodiments of the present invention, the calixarene is bonded to the surface of the intermittent catheter via one or more surface-linker groups on a rim of the calixarene, said surface-linker groups being bonded to the surface of the device via covalent bonds, ionic bonds, hydrogen bonds, or Van der Waals forces. In some embodiments, an opposing rim of the calixarene is substituted by one or more polyethylene glycol, polypropylene glycol or polytrimethylene glycol groups, or a mixture thereof, which form the surface linker group.

In some embodiments, said glycol linker groups are attached to the calixarene via (C₁-C₃₀)alkylene spacer groups, said glycol groups, each independently, have from 2 to 250 repeating glycol units and may be optionally terminated by hydrogen or (C₁ to C₄)alkyl. The (C₁ to C₃₀)alkylene spacer groups may be optionally substituted by one or more fluoro, methyl or ethyl groups and may optionally contain one or more unsaturated bonds.

Preferably said glycol groups are attached to the calixarene via (C₃-C₁₆)alkylene spacer groups.

In some embodiments, said glycol groups are attached to the alkylene spacer group directly via the oxygen of the glycol or via another linker group.

In some embodiments, the glycol linker group is selected from carbonate, carbamate, urea, phosphate and triazole.

In some embodiments, the calixarene is bonded to the surface of the intermittent catheter via 2 to 8 surface-linker groups.

In some embodiments, the calixarene is bonded to the surface of the intermittent catheter via 2 or 4 surface-linker groups.

In some embodiments, said surface-linker groups are bonded to the surface of the device via covalent bonds.

In some embodiments, a rim of the calixarene is substituted by one or more polyethylene glycol groups.

In some embodiments, the calixarene is derived from a phenol, a resorcinol or a pyrogallol, or mixtures thereof.

In some embodiments, said calixarene is derived from a compound of formula (I)

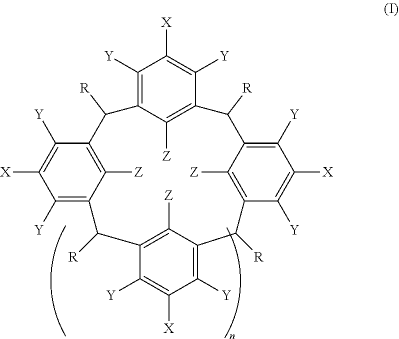

Preferably X is H, (C₁-C₃₀)alkyl, NH₂, NH(C₁-C₃₀)alkyl, N(C₁-C₃₀)alkyl₂, CH₂NH(C₁-C₃₀)alkyl, OH, O(C₁-C₃₀)alkyl or OCH₂CO₂(C₁-C₃₀)alkyl;

Y is OH, O(C₁-C₃₀)alkyl, OCH₂CO₂(C₁-C₃₀)alkyl, H, (C₁-C₃₀)alkyl, NH₂, NH(C₁-C₃₀)alkyl, N(C₁-C₃₀)alkyl₂ or CH₂NH(C₁-C₃₀)alkyl;

Z is H, OH or methyl;

n is 1, 3 or 5; and

R is —(C₁-C₃₀)alkyl- or —(C₁-C₃₀)alkylene-L¹-G-R¹ wherein said alkyl or alkylene may be optionally substituted by one or more fluoro, methyl or ethyl groups and may optionally contain one or more unsaturated bonds;

L¹ is a bond or a linking group;

G is —O(CH₂CH₂O)ₘ-, —O(CH₂CH(CH₃)O)ₘ-, —(O(CH(CH₃)CH₂O)ₘ-, or —O(CH₂CH₂C₂O)ₘ-;

m is 2 to 250;

R¹ is H or (C₁-C₃₀)alkyl;

and wherein each X, Y, Z, R and R¹ group may be the same or different.

In some embodiments there may be more than one calixarene independently selected from the calixarenes of formula (I), such as two, three or four independently selected calixarenes.

In some embodiments, X is (C₁-C₂₅)alkyl, (C₁-C₂₀)alkyl, (C₁-C₁₅)alkyl, (C₁-C₁₀)alkyl, (C₁-C₅)alkyl or preferably (C₁-C₄)alkyl.

In some embodiments, X is (C₅-C₃₀)alkyl, (C₁₀-C₃₀)alkyl, (C₁₅-C₃₀)alkyl, (C₁₅-C₃₀)alkyl, (C₂₀-C₃₀)alkyl or (C₂₅-C₃₀)alkyl.

In further embodiments X is (C₅-C₂₅)alkyl, (C₁₀-C₂₅)alkyl, (C₁₅-C₂₅)alkyl, (C₂₀-C₂₅)alkyl, (C₅-C₂₀)alkyl, (C₅-C₁₅)alkyl, (C₅-C₁₀)alkyl, (C₁₀-C₂₀)alkyl, (C₁₅-C₂₀)alkyl or (C₁₀-C₁₅)alkyl.

In preferred embodiments, X is (C₁-C₅)alkyl, especially (C₁-C₄)alkyl.

In some embodiments, X is NH₂.

In some embodiments, X is NH(C₁-C₂₅)alkyl, NH(C₁-C₂₀)alkyl, NH(C₁-C₁₅)alkyl, NH(C₁-C₁₀)alkyl, NH(C₁-C₅)alkyl or NH(C₁-C₄)alkyl.

In some embodiments, X is NH(C₅-C₃₀)alkyl, NH(C₁₀-C₃₀)alkyl, NH(C₁₅-C₃₀)alkyl, NH(C₁₅-C₃₀)alkyl, NH(C₂₀-C₃₀)alkyl or NH(C₂₅-C₃₀)alkyl.

In further embodiments X is NH(C₅-C₂₅)alkyl, NH(C₁₀-C₂₅)alkyl, NH(C₁₅-C₂₅)alkyl, NH(C₂₀-C₂₅)alkyl, NH(C₅-C₂₀)alkyl, NH(C₅-C₁₅)alkyl, NH(C₅-C₁₀)alkyl, NH(C₁₀-C₂₀)alkyl, NH(C₁₅-C₂₀)alkyl or NH(C₁₀-C₁₅)alkyl.

In preferred embodiments, X is NH(C₁-C₅)alkyl, especially NH(C₁-C₄)alkyl.

In some embodiments, X is N(C₁-C₂₅)alkyl₂, N(C₁-C₂₀)alkyl₂, N(C₁-C₁₅)alkyl₂, N(C₁-C₁₀)alkyl₂, N(C₁-C₅)alkyl₂ or N(C₁-C₄)alkyl₂.

In some embodiments, X is N(C₅-C₃₀)alkyl₂, N(C₁₀-C₃₀)alkyl₂, N(C₁₅-C₃₀)alkyl₂, N(C₁₅-C₃₀)alkyl₂, N(C₂₀-C₃₀)alkyl₂ or N(C₂₅-C₃₀)alkyl₂.

In further embodiments X is N(C₅-C₂₅)alkyl₂, N(C₁₀-C₂₅)alkyl₂, N(C₁₅-C₂₅)alkyl₂, N(C₂₀-C₂₅)alkyl₂, N(C₅-C₂₀)alkyl₂, N(C₅-C₁₅)alkyl₂, N(C₅-C₁₀)alkyl₂, N(C₁₀-C₂₀)alkyl₂, N(C₁₅-C₂₀)alkyl₂ or N(C₁₀-C₁₅)alkyl₂.

In preferred embodiments, X is N(C₁-C₅)alkyl₂, especially N(C₁-C₄)alkyl₂.

In some embodiments, X is CH₂NH(C₁-C₂₅)alkyl, CH₂NH(C₁-C₂₀)alkyl, CH₂NH(C₁-C₁₅)alkyl, CH₂NH(C₁-C₁₀)alkyl, CH₂NH(C₁-C₅)alkyl or CH₂NH(C₁-C₄)alkyl.

In some embodiments, X is CH₂NH(C₅-C₃₀)alkyl, CH₂NH(C₁₀-C₃₀)alkyl, CH₂NH(C₁₅-C₃₀)alkyl, CH₂NH(C₁₅-C₃₀)alkyl, CH₂NH(C₂₀-C₃₀)alkyl or CH₂NH(C₂₅-C₃₀)alkyl.

In further embodiments X is CH₂NH(C₅-C₂₅)alkyl, CH₂NH(C₁₀-C₂₅)alkyl, CH₂NH(C₁₅-C₂₅)alkyl, CH₂NH(C₂₀-C₂₅)alkyl, CH₂NH(C₅-C₂₀)alkyl, CH₂NH(C₅-C₁₅)alkyl, CH₂NH(C₅-C₁₀)alkyl, CH₂NH(C₁₀-C₂₀)alkyl, CH₂NH(C₁₅-C₂₀)alkyl or CH₂NH(C₁₀-C₁₅)alkyl.

In preferred embodiments, X is CH₂NH(C₁-C₅)alkyl, especially CH₂NH(C₁-C₄)alkyl.

In some embodiments, X is OH.

In some embodiments, X is O(C₁-C₂₅)alkyl, O(C₁-C₂₀)alkyl, O(C₁-C₁₅)alkyl, O(C₁-C₁₀)alkyl, O(C₁-C₅)alkyl or O(C₁-C₄)alkyl.

In some embodiments, X is O(C₅-C₃₀)alkyl, O(C₁₀-C₃₀)alkyl, O(C₁₅-C₃₀)alkyl, O(C₁₅-C₃₀)alkyl, O(C₂₀-C₃₀)alkyl or O(C₂₅-C₃₀)alkyl.

In further embodiments X is O(C₅-C₂₅)alkyl, O(C₁₀-C₂₅)alkyl, O(C₁₅-C₂₅)alkyl, O(C₂₀-C₂₅)alkyl, O(C₅-C₂₀)alkyl, O(C₅-C₁₅)alkyl, O(C₅-C₁₀)alkyl, O(C₁₀-C₂₀)alkyl, O(C₁₅-C₂₀)alkyl or O(C₁₀-C₁₅)alkyl.

In preferred embodiments, X is O(C₁-C₅)alkyl, especially O(C₁-C₄)alkyl.

In some embodiments, X is OCH₂CO₂(C₁-C₂₅)alkyl, OCH₂CO₂(C₁-C₂₀)alkyl, OCH₂CO₂(C₁-C₁₅)alkyl, OCH₂CO₂(C₁-C₁₀)alkyl, OCH₂CO₂(C₁-C₅)alkyl or OCH₂CO₂(C₁-C₄)alkyl.

In some embodiments, X is OCH₂CO₂(C₅-C₃₀)alkyl, OCH₂CO₂(C₁₀-C₃₀)alkyl, OCH₂CO₂(C₁₅-C₃₀)alkyl, OCH₂CO₂(C₁₅-C₃₀)alkyl, OCH₂CO₂(C₂₀-C₃₀)alkyl or OCH₂CO₂(C₂₅-C₃₀)alkyl.

In further embodiments X is OCH₂CO₂(C₅-C₂₅)alkyl, OCH₂CO₂(C₁₀-C₂₅)alkyl, OCH₂CO₂(C₁₅-C₂₅)alkyl, OCH₂CO₂(C₂₀-C₂₅)alkyl, OCH₂CO₂(C₅-C₂₀)alkyl, OCH₂CO₂(C₅-C₁₅)alkyl, OCH₂CO₂(C₅-C₁₀)alkyl, OCH₂CO₂(C₁₀-C₂₀)alkyl, OCH₂CO₂(C₁₅-C₂₀)alkyl or OCH₂CO₂(C₁₀-C₁₅)alkyl.

In preferred embodiments, X is OCH₂CO₂(C₁-C₅)alkyl, especially OCH₂CO₂(C₁-C₄)alkyl.

In other preferred embodiments, X is H.

In some embodiments, Y is O(C₁-C₂₅)alkyl, O(C₁-C₂₀)alkyl, O(C₁-C₁₅)alkyl, O(C₁-C₁₀)alkyl, O(C₁-C₅)alkyl or O(C₁-C₄)alkyl.

In some embodiments, Y is O(C₅-C₃₀)alkyl, O(C₁₀-C₃₀)alkyl, O(C₁₅-C₃₀)alkyl, O(C₁₅-C₃₀)alkyl, O(C₂₀-C₃₀)alkyl or O(C₂₅-C₃₀)alkyl.

In further embodiments Y is O(C₅-C₂₅)alkyl, O(C₁₀-C₂₅)alkyl, O(C₁₅-C₂₅)alkyl, O(C₂₀-C₂₅)alkyl, O(C₅-C₂₀)alkyl, O(C₅-C₁₅)alkyl, O(C₅-C₁₀)alkyl, O(C₁₀-C₂₀)alkyl, O(C₁₅-C₂₀)alkyl or O(C₁₀-C₁₅)alkyl.

In preferred embodiments, Y is O(C₁-C₅)alkyl, especially O(C₁-C₄)alkyl.

In some embodiments, Y is OCH₂CO₂(C₁-C₂₅)alkyl, OCH₂CO₂(C₁-C₂₀)alkyl, OCH₂CO₂(C₁-C₁₅)alkyl, OCH₂CO₂(C₁-C₁₀)alkyl, OCH₂CO₂(C₁-C₅)alkyl or OCH₂CO₂(C₁-C₄)alkyl.

In some embodiments, Y is OCH₂CO₂(C₅-C₃₀)alkyl, OCH₂CO₂(C₁₀-C₃₀)alkyl, OCH₂CO₂(C₁₅-C₃₀)alkyl, OCH₂CO₂(C₁₅-C₃₀)alkyl, OCH₂CO₂(C₂₀-C₃₀)alkyl or OCH₂CO₂(C₂₅-C₃₀)alkyl.

In further embodiments Y is OCH₂CO₂(C₅-C₂₅)alkyl, OCH₂CO₂(C₁₀-C₂₅)alkyl, OCH₂CO₂(C₁₅-C₂₅)alkyl, OCH₂CO₂(C₂₀-C₂₅)alkyl, OCH₂CO₂(C₅-C₂₀)alkyl, OCH₂CO₂(C₅-C₁₅)alkyl, OCH₂CO₂(C₅-C₁₀)alkyl, OCH₂CO₂(C₁₀-C₂₀)alkyl, OCH₂CO₂(C₁₅-C₂₀)alkyl or OCH₂CO₂(C₁₀-C₁₅)alkyl.

In preferred embodiments, Y is OCH₂CO₂(C₁-C₅)alkyl, especially OCH₂CO₂(C₁-C₄)alkyl.

In some embodiments, Y is H.

In some embodiments, Y is (C₁-C₂₅)alkyl, (C₁-C₂₀)alkyl, (C₁-C₁₅)alkyl, (C₁-C₁₀)alkyl, (C₁-C₅)alkyl or (C₁-C₄)alkyl. In some embodiments, Y is (C₅-C₃₀)alkyl, (C₁₀-C₃₀)alkyl, (C₁₅-C₃₀)alkyl, (C₁₅-C₃₀)alkyl, (C₂₀-C₃₀)alkyl or (C₂₅-C₃₀)alkyl. In further embodiments Y is (C₅-C₂₅)alkyl, (C₁₀-C₂₅)alkyl, (C₁₅-C₂₅)alkyl, (C₂₀-C₂₅)alkyl, (C₅-C₂₀)alkyl, (C₅-C₁₅)alkyl, (C₅-C₁₀)alkyl, (C₁₀-C₂₀)alkyl, (C₁₅-C₂₀)alkyl or (C₁₀-C₁₅)alkyl.

In preferred embodiments, Y is (C₁-C₅)alkyl, especially (C₁-C₄)alkyl.

In some embodiments, Y is NH₂.

In some embodiments, Y is NH(C₁-C₂₅)alkyl, NH(C₁-C₂₀)alkyl, NH(C₁-C₁₅)alkyl, NH(C₁-C₁₀)alkyl, NH(C₁-C₅)alkyl or NH(C₁-C₄)alkyl. In some embodiments, Y is NH(C₅-C₃₀)alkyl, NH(C₁₀-C₃₀)alkyl, NH(C₁₅-C₃₀)alkyl, NH(C₁₅-C₃₀)alkyl, NH(C₂₀-C₃₀)alkyl or NH(C₂₅-C₃₀)alkyl. In further embodiments Y is NH(C₅-C₂₅)alkyl, NH(C₁₀-C₂₅)alkyl, NH(C₁₅-C₂₅)alkyl, NH(C₂₀-C₂₅)alkyl, NH(C₅-C₂₀)alkyl, NH(C₅-C₁₅)alkyl, NH(C₅-C₁₀)alkyl, NH(C₁₀-C₂₀)alkyl, NH(C₁₅-C₂₀)alkyl or NH(C₁₀-C₁₅)alkyl.

In preferred embodiments, Y is NH(C₁-C₅)alkyl, especially NH(C₁-C₄)alkyl.

In some embodiments, Y is N(C₁-C₂₅)alkyl₂, N(C₁-C₂₀)alkyl₂, N(C₁-C₁₅)alkyl₂, N(C₁-C₁₀)alkyl₂, N(C₁-C₅)alkyl₂ or N(C₁-C₄)alkyl₂. In some embodiments, Y is N(C₅-C₃₀)alkyl₂, N(C₁₀-C₃₀)alkyl₂, N(C₁₅-C₃₀)alkyl₂, N(C₁₅-C₃₀)alkyl₂, N(C₂₀-C₃₀)alkyl₂ or N(C₂₅-C₃₀)alkyl₂. In further embodiments Y is N(C₅-C₂₅)alkyl₂, N(C₁₀-C₂₅)alkyl₂, N(C₁₅-C₂₅)alkyl₂, N(C₂₀-C₂₅)alkyl₂, N(C₅-C₂₀)alkyl₂, N(C₅-C₁₅)alkyl₂, N(C₅-C₁₀)alkyl₂, N(C₁₀-C₂₀)alkyl₂, N(C₁₅-C₂₀)alkyl₂ or N(C₁₀-C₁₅)alkyl₂. In preferred embodiments, Y is N(C₁-C₅)alkyl₂, especially N(C₁-C₄)alkyl₂.

In some embodiments, Y is CH₂NH(C₁-C₂₅)alkyl, CH₂NH(C₁-C₂₀)alkyl, CH₂NH(C₁-C₁₅)alkyl, CH₂NH(C₁-C₁₀)alkyl, CH₂NH(C₁-C₅)alkyl or CH₂NH(C₁-C₄)alkyl.

In some embodiments, Y is CH2NH(C₅-C₃₀)alkyl, CH2NH(C₁₀-C₃₀)alkyl, CH₂NH(C₁₅-C₃₀)alkyl, CH2NH(C₁₅-C₃₀)alkyl, CH₂NH(C₂₀-C₃₀)alkyl or CH₂NH(C₂₅-C₃₀)alkyl.

In further embodiments Y is CH2NH(C₅-C₂₅)alkyl, CH2NH(C₁₀-C₂₅)alkyl,

CH₂NH(C₁₅-C₂₅)alkyl, CH₂NH(C₂₀-C₂₅)alkyl, CH₂NH(C₅-C₂₀)alkyl, CH₂NH(C₅-C₁₅)alkyl, CH₂NH(C₅-C₁₀)alkyl, CH₂NH(C₁₀-C₂₀)alkyl, CH₂NH(C₁₅-C₂₀)alkyl or CH₂NH(C₁₀-C₁₅)alkyl.

In preferred embodiments, Y is CH₂NH(C₁-C₅)alkyl, especially CH₂NH(C₁-C₄)alkyl.

In other preferred embodiments, Y is OH

In some embodiments, Z is OH.

In some embodiments, Z is methyl.

In preferred embodiments, Z is H.

In some embodiments, n is 3.

In some embodiments, n is 5.

In preferred embodiments, n is 1.

In some embodiments, R is (C₁-C₂₅)alkyl, (C₁-C₂₀)alkyl, (C₁-C₁₅)alkyl, (C₁-C₁₀)alkyl or (C₁-C₅)alkyl, wherein said alkyl may be substituted by one or more fluoro, methyl or ethyl groups.

In some embodiments, R is (C₅-C₃₀)alkyl, (C₁₀-C₃₀)alkyl, (C₁₅-C₃₀)alkyl, (C₁₅-C₃₀)alkyl, (C₂₀-C₃₀)alkyl or (C₂₅-C₃₀)alkyl, wherein said alkyl may be substituted by one or more fluoro, methyl or ethyl groups.

In further embodiments R is (C₅-C₂₅)alkyl, (C₁₀-C₂₅)alkyl, (C₁₅-C₂₅)alkyl, (C₂₀-C₂₅)alkyl, (C₅-C₂₀)alkyl, (C₅-C₁₅)alkyl, (C₅-C₁₀)alkyl, (C₁₀-C₂₀)alkyl, (C₁₅-C₂₀)alkyl, (C₁₀-C₁₅)alkyl or (C₃-C₁₆)alkyl, wherein said alkyl may be substituted by one or more fluoro, methyl or ethyl groups.

In preferred embodiments, R is (C₁-C₂₀)alkyl, especially (C₃-C₁₆)alkyl, wherein said alkyl may be substituted by one or more fluoro, methyl or ethyl groups.

In some embodiments, R is (C₁-C₂₅)alkylene-L¹-G-R¹, (C₁-C₂₀)alkylene-L¹-G-R¹, (C₁-C₁₅)alkylene-L¹-G-R¹, (C₁-C₁₀)alkylene-L¹-G-R¹ or (C₁-C₅)alkylene-L¹-G-R¹, wherein said alkyl may be substituted by one or more fluoro, methyl or ethyl groups.

In some embodiments, R is (C₅-C₃₀)alkylene-C-G-R¹, (C₁₀-C₃₀)alkylene-C-G-R¹, (C₁₅-C₃₀)alkylene-L¹-G-R¹, (C₁₅-C₃₀)alkylene-L¹-G-R¹, (C₂₀-C₃₀)alkylene-C-G-R¹ or (C₂₅-C₃₀)alkylene-C-G-R¹, wherein said alkyl may be substituted by one or more fluoro, methyl or ethyl groups.

In further embodiments, R is (C₅-C₂₅)alkylene-L¹-G-R¹, (C₁₀-C₂₅)alkylene-L¹-G-R¹, (C₁₅-C₂₅)alkylene-L¹-G-R¹, (C₂₀-C₂₅)alkylene-L¹-G-R¹, (C₅-C₂₀)alkylene-C-G-R¹, (C₅-C₁₅)alkylene-L¹-G-R¹, (C₅-C₁₀)alkylene-C-G-R¹, (C₁₀-C₂₀)alkylene-L¹-G-R¹, (C₁₅-C₂₀)alkylene-L¹-G-R¹, (C₁₀-C₁₅)alkylene-L¹-G-R¹ or (C₃-C₁₆)alkylene-L¹-G-R¹, wherein said alkyl may be substituted by one or more fluoro, methyl or ethyl groups.

In preferred embodiments, R is (C₁-C₂₀)alkylene-L¹-G-R¹, especially (C₃-C₁₆)alkylene-L¹-G-R¹, wherein said alkyl may be substituted by one or more fluoro, methyl or ethyl groups.

In preferred embodiments, R is —(C₁₀)alkylene-L¹-G-R¹.

In preferred embodiments, R is —CH₂C₈F₁₇.

In some embodiments, L¹ is a bond.

In some embodiments, L¹ is a linking group selected from carbonate, carbamate, urea, phosphate and triazole.

In preferred embodiments, L¹ is carbonate.

In some embodiments, G is —O(CH₂CH(CH₃)O)ₘ-.

In some embodiments, G is —(O(CH(CH₃)CH₂O)ₘ-.

In some embodiments, G is —O(CH₂CH₂CH₂O)ₘ-.

In preferred embodiments, G is —O(CH₂CH₂O)ₘ-.

In some embodiments, m is 2 to 225, 2 to 200, 2 to 175, 2 to 150, 2 to 125, 2 to 100, 2 to 75, 2 to 50 or 2 to 25.

In some embodiments, m is 5 to 250, 25 to 250, 50 to 250, 75 to 250, 100 to 250, 125 to 250, 150 to 250, 175 to 250, 200 to 250, 225 to 250.

In preferred embodiments, m is 3 to 150, especially 6 to 50, and especially 15 to 25.

In some embodiments, R¹ is (C₁-C₂₅)alkyl, (C₁-C₂₀)alkyl, (C₁-C₁₅)alkyl, (C₁-C₁₀)alkyl, (C₁-C₅)alkyl or (C₁-C₄)alkyl.

In some embodiments, R¹ is (C₅-C₃₀)alkyl, (C₁₀-C₃₀)alkyl, (C₁₅-C₃₀)alkyl, (C₁₅-C₃₀)alkyl, (C₂₀-C₃₀)alkyl or (C₂₅-C₃₀)alkyl.

In further embodiments R¹ is (C₅-C₂₅)alkyl, (C₁₀-C₂₅)alkyl, (C₁₅-C₂₅)alkyl, (C₂₀-C₂₅)alkyl, (C₅-C₂₀)alkyl, (C₅-C₁₅)alkyl, (C₅-C₁₀)alkyl, (C₁₀-C₂₀)alkyl, (C₁₅-C₂₀)alkyl or (C₁₀-C₁₅)alkyl.

In preferred embodiments, R¹ is (C₁-C₅)alkyl, especially (C₁-C₄)alkyl, and especially methyl.

In other preferred embodiments, R¹ is H.

In preferred embodiments, X is (C₁-C₅)alkyl, especially (C₁-C₄)alkyl; and Y is O(C₁-C₅)alkyl, especially O(C₁-C₄)alkyl.

In preferred embodiments, X is (C₁-C₅)alkyl, especially (C₁-C₄)alkyl; and Y is OCH₂CO₂(C₁-C₅)alkyl, especially OCH₂CO₂(C₁-C₄)alkyl.

In preferred embodiments, Y is OH; and X is (C₁-C₅)alkyl, especially (C₁-C₄)alkyl.

In preferred embodiments, Y is OH; and X is NH(C₁-C₅)alkyl, especially NH(C₁-C₄)alkyl.

In preferred embodiments, Y is OH; and X is N(C₁-C₅)alkyl₂, especially N(C₁-C₄)alkyl₂.

In preferred embodiments, Y is OH; and X is CH₂NH(C₁-C₅)alkyl, especially CH₂NH(C₁-C₄)alkyl.

In preferred embodiments, Y is OH; and X is NH₂.

In preferred embodiments, Y is OH; and X is OH.

In preferred embodiments, Y is OH; and X is O(C₁-C₅)alkyl, especially O(C₁-C₄)alkyl.

In preferred embodiments, Y is OH; and X is OCH₂CO₂(C₁-C₅)alkyl, especially OCH₂CO₂(C₁-C₄)alkyl.

In preferred embodiments, Y is OH; and X is H.

In preferred embodiments, X is H; and Y is O(C₁-C₅)alkyl, especially O(C₁-C₄)alkyl.

In preferred embodiments, X is H; and Y is OCH₂CO₂(C₁-C₅)alkyl, especially OCH₂CO₂(C₁-C₄)alkyl.

In preferred embodiments, X is NH₂; and Y is O(C₁-C₅)alkyl, especially O(C₁-C₄)alkyl.

In preferred embodiments, X is NH2; and Y is OCH₂CO₂(C₁-C₅)alkyl, especially OCH₂CO₂(C₁-C₄)alkyl.

In preferred embodiments, X is NH(C₁-C₅)alkyl, especially NH(C₁-C₄)alkyl; and Y is O(C₁-C₅)alkyl, especially O(C₁-C₄)alkyl.

In preferred embodiments, X is NH(C₁-C₅)alkyl, especially NH(C₁-C₄)alkyl; and Y is OCH₂CO₂(C₁-C₅)alkyl, especially OCH₂CO₂(C₁-C₄)alkyl.

In preferred embodiments, X is CH₂NH(C₁-C₅)alkyl, especially CH₂NH(C₁-C₄)alkyl; and Y is O(C₁-C₅)alkyl, especially O(C₁-C₄)alkyl.

In preferred embodiments, X is CH₂NH(C₁-C₅)alkyl, especially CH₂NH(C₁-C₄)alkyl; and Y is OCH2CO2(C₁-C₅)alkyl, especially OCH₂CO₂(C₁-C₄)alkyl.

In preferred embodiments, X is N(C₁-C₅)alkyl2, especially N(C₁-C₄)alkyl₂; and Y is O(C₁-C₅)alkyl, especially O(C₁-C₄)alkyl.

In preferred embodiments, X is N(C₁-C₅)alkyl₂, especially N(C₁-C₄)alkyl₂; and Y is OCH2CO2(C₁-C₅)alkyl, especially OCH₂CO₂(C₁-C₄)alkyl.

In preferred embodiments, X is OH; and Y is H.

In preferred embodiments, X is OH; and Y is (C₁-C₅)alkyl, especially (C₁-C₄)alkyl.

In some embodiments, X is OH; and Y is NH₂.

In preferred embodiments, X is OH; and Y is NH(C₁-C₅)alkyl, especially NH(C₁-C₄)alkyl.

In preferred embodiments, X is OH; and Y is N(C₁-C₅)alkyl₂, especially N(C₁-C₄)alkyl₂.

In preferred embodiments, X is OH; and Y is CH₂NH(C₁-C₅)alkyl, especially CH₂NH(C₁-C₄)alkyl.

In preferred embodiments, X is OH; and Y is O(C₁-C₅)alkyl, especially O(C₁-C₄)alkyl.

In preferred embodiments, X is OH; and Y is OCH₂CO₂(C₁-C₅)alkyl, especially OCH₂CO₂(C₁-C₄)alkyl.

In preferred embodiments, X is O(C₁-C₅)alkyl, especially O(C₁-C₄)alkyl; and Y is (C₁-C₅)alkyl, especially (C₁-C₄)alkyl.

In preferred embodiments, X is O(C₁-C₅)alkyl, especially O(C₁-C₄)alkyl; and Y is OCH₂CO₂(C₁-C₅)alkyl, especially OCH₂CO₂(C₁-C₄)alkyl.

In preferred embodiments, X is O(C₁-C₅)alkyl, especially O(C₁-C₄)alkyl; and Y is O(C₁-C₅)alkyl, especially O(C₁-C₄)alkyl.

In preferred embodiments, X is O(C₁-C₅)alkyl, especially O(C₁-C₄)alkyl; and Y is NH(C₁-C₅)alkyl, especially NH(C₁-C₄)alkyl.

In preferred embodiments, X is O(C₁-C₅)alkyl₂, especially O(C₁-C₄)alkyl₂; and Y is N(C₁-C₅)alkyl₂, especially N(C₁-C₄)alkyl₂.

In preferred embodiments, X is O(C₁-C₅)alkyl, especially O(C₁-C₄)alkyl; and Y is CH₂NH(C₁-C₅)alkyl, especially CH₂NH(C₁-C₄)alkyl.

In preferred embodiments, X is OCH₂CO₂(C₁-C₅)alkyl, especially OCH₂CO₂(C₁-C₄)alkyl; and Y is (C₁-C₅)alkyl, especially (C₁-C₄)alkyl.

In preferred embodiments, X is OCH₂CO₂(C₁-C₅)alkyl, especially OCH₂CO₂(C₁-C₄)alkyl; and Y is NH(C₁-C₅)alkyl, especially NH(C₁-C₄)alkyl.

In preferred embodiments, X is OCH₂CO₂(C₁-C₅)alkyl, especially OCH₂CO₂(C₁-C₄)alkyl; and Y is N(C₁-C₅)alkyl₂, especially N(C₁-C₄)alkyl₂.

In preferred embodiments, X is OCH₂CO₂(C₁-C₅)alkyl, especially OCH₂CO₂(C₁-C₄)alkyl; and Y is CH₂NH(C₁-C₅)alkyl, especially CH₂NH(C₁-C₄) alkyl.

In preferred embodiments, X is OCH₂CO₂(C₁-C₅)alkyl, especially OCH₂CO₂(C₁-C₄)alkyl; and Y is O(C₁-C₅)alkyl, especially O(C₁-C₄)alkyl.

In preferred embodiments, X is OCH₂CO₂(C₁-C₅)alkyl, especially OCH₂CO₂(C₁-C₄)alkyl; and Y is OCH₂CO₂(C₁-C₅)alkyl, especially OCH₂CO₂(C₁-C₄)alkyl.

In preferred embodiments, Y is H; and X is O(C₁-C₅)alkyl, especially O(C₁-C₄)alkyl.

In preferred embodiments, Y is H; and X is OCH₂CO₂(C₁-C₅)alkyl, especially OCH₂CO₂(C₁-C₄)alkyl.

In preferred embodiments, Y is NH₂; and X is O(C₁-C₅)alkyl, especially O(C₁-C₄)alkyl.

In preferred embodiments, Y is NH₂; and X is OCH₂CO₂(C₁-C₅)alkyl, especially OCH₂CO₂(C₁-C₄)alkyl.

In some preferred embodiments, X is H and R is —CH₂C₈F₁₇.

In a preferred embodiment, X is H, Y is OH, Z is H, n is 1, and R is —CH₂CH₂CH₂CH₂CH₂CH₂CH₂CH₂CH═CH₂.

In a preferred embodiment, X is H, Y is OBoc, Z is H, n is 1, and R is —CH₂CH₂CH₂CH₂CH₂CH₂CH₂CH₂CH═CH₂.

In a preferred embodiment, X is H, Y is OBoc, Z is H, n is 1, and R is —CH₂CH₂CH₂CH₂CH₂CH₂CH₂CH₂CH₂CH₂OH.

In a preferred embodiment, X is H, Y is OH, Z is H, n is 1, and R is —CH₂CH₂CH₂CH₂CH₂CH₂CH₂CH₂CH₂CH₂OH.

In some embodiments, the calixarene is bonded to the surface of the device via surface-linker groups substituted for any one or more of the X or Y substituents, or a combination thereof.

In some embodiments, the surface-linker groups X or Y, or a combination thereof, on the calixarene are derived from acid chloride, chloroformate, vinyl, acrylate, methacrylate, ethacrylate, or silane functional groups.

In some embodiments, the surface-linker groups are derived from silane functional groups, which form one or more siloxane bonds with a device having a silicone surface.

In some embodiments, the surface-linker groups X and/or Y are L²-Si(R²)₃, or X and an adjacent Y group together form

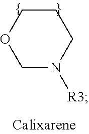

L² is a spacer group;

R³ is (C₂-C₁₀)alkylene-Si(R²)₃, wherein said alkylene may be optionally substituted by one or more fluoro, methyl or ethyl groups and may optionally contain one or more unsaturated bonds; Si(R²)₃ is selected from Si[O(C₁-C₄)alkyl]₃, SiCl₃, Si[(C₁-C₄)alkyl]₂Cl and Si[(C₁-C₄)alkyl]Cl₂;

each alkyl and each surface-linker group may be the same or different; and

said silane providing functionality for bonding to the surface of the catheter.

In some embodiments, L² is selected from O(C₂-C₁₀)alkylene, CH₂NH(C₂-C₁₀)alkylene, OCH₂CO₂(C₂-C₁₀)alkylene and OCH₂CONH(C₂-C₁₀)alkylene, wherein said alkylene may be optionally substituted by one or more fluoro, methyl or ethyl groups and may optionally contain one or more unsaturated bonds.

In some embodiments, when X and Y are OH, Z is H, and n is 1; R is not —(C₃)alkylene—O(CH₂CH₂O)₄CH₃.

In some embodiments, R is —(C₃-C₁₆)alkylene-OH or —(C₂-C₁₅)alkylene-CH₂═CH₂ wherein said alkylene may be optionally substituted by one or more fluoro, methyl or ethyl groups and may optionally contain one or more unsaturated bonds;

and each X, Y, Z and R group may be the same or different; with the proviso that when X is H, OH, CH₃ or OCH₃; Y is OH; Z is H; and n is 1; R is not —(C₈)alkylene-CH₂═CH₂.

In some embodiments, the calixarene is

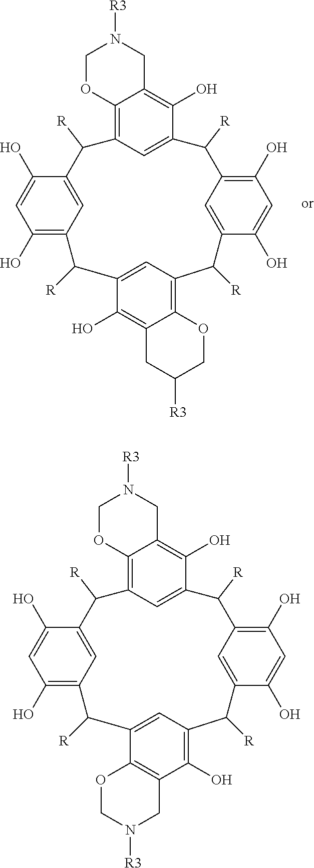

In some embodiments, R is —(C₁₀)alkylene-OC(O)O—(CH₂CH₂O)ₘ—OH; and R³ is (C₃)alkylene-Si(OEt)₃ or a combination thereof.

In a preferred embodiment, R is —CH₂CH₂CH₂CH₂CH₂CH₂CH₂CH₂CH₂CH₂OH, and R³ is —CH₂CH₂CH₂Si(OEt)₃.

In a preferred embodiment, R is —CH₂CH₂CH₂CH₂CH₂CH₂CH₂CH₂CH₂CH₂OC(O)O—(CH₂CH₂O)ₘ—CH₃, and R³ is —CH₂CH₂CH₂Si(OEt)₃.

In a preferred embodiment, R is —CH₂CH₂CH₂CH₂CH₂CH₂CH₂CH₂CH₂CH₂OC(O)O—(CH₂CH₂O)₇—CH₃, and R³ is —CH₂CH₂CH₂Si(OEt)₃.

In some embodiments, the intermittent catheter is formed of a material of the group comprising: polyvinyl chloride, polytetrafluoroethylene, polyolefins, latex, silicones, synthetic rubbers, polyurethanes, polyesters, polyacrylates, polyamides, thermoplastic elastomeric materials, styrene block copolymers, polyether block amide, thermoplastic vulcanizates, thermoplastic copolyesters, thermoplastic polyamides, and water disintegrable or enzymatically hydrolysable material, or combinations, blends or co-polymers of any of the above materials.

In preferred embodiments, the intermittent catheter is formed of a material of the group comprising: polyolefins, polyesters, polyacrylates, polyamides, thermoplastic elastomeric material, polyether block amide, thermoplastic vulcanizates, thermoplastic copolyesters, thermoplastic polyamides, fluororubber, and water disintegrable or enzymatically hydrolysable material or combinations, blends or co-polymers of any of the above materials.

In some embodiments, said water disintegrable or enzymatically hydrolysable material comprises a material of teh group comprising: polyvinyl alcohol, extrudable polyvinyl alcohol, polyacrylic acids, polylactic acid, polyesters, polyglycolide, polyglycolic acid, poly lactic-co-glycolic acid, polylactide, amines, polyacrylamides, poly(N-(2-Hydroxypropyl) methacrylamide), starch, modified starches or derivatives, amylopectin, pectin, xanthan, scleroglucan, dextrin, chitosans, chitins, agar, alginate, carrageenans, laminarin, saccharides, polysaccharides, sucrose, polyethylene oxide, polypropylene oxide, acrylics, polyacrylic acid blends, poly(methacrylic acid), polystyrene sulfonate, polyethylene sulfonate, lignin sulfonate, polymethacrylamides, copolymers of aminoalkyl-acrylamides and methacrylamides, melamine-formaldehyde copolymers, vinyl alcohol copolymers, cellulose ethers, poly-ethers, polyethylene oxide, blends of polyethylene-polypropylene glycol, carboxymethyl cellulose, guar gum, locust bean gum, hydroxypropyl cellulose, vinylpyrrolidone polymers and copolymers, polyvinyl pyrrolidone-ethylene-vinyl acetate, polyvinyl pyrrolidone-carboxymethyl cellulose, carboxymethyl cellulose shellac, copolymers of vinylpyrrolidone with vinyl acetate, hydroxyethyl cellulose, gelatin, poly-caprolactone, poly(p-dioxanone), or combinations, blends or co-polymers of any of the above materials.

In some preferred embodiments, the intermittent catheter is formed of a polyolefin material, especially polyethylene and/or polypropylene.

In some preferred embodiments, the intermittent catheter is formed of a thermoplastic elastomeric material.

In some embodiments, the surface of the intermittent catheter is prepared prior to bonding of a calixarene to the surface. The preparation may comprise one or more of the group comprising: corona treatment, plasma treatment, and flame treatment, which methods are particularly useful for polyolefin catheter materials and surfaces. The preparation may comprise surface oxidation, which may comprise cleavage of polymer chains on the surface of the intermittent catheter and incorporation of carbonyl and/or hydroxyl functional groups.

The calixarene may be modified to comprise pendant groups, which may comprise vinyl and/or acrylate groups for grafting of the calixarene to the surface of the intermittent catheter post-preparation of the surface, which in some embodiments are polyolefin surfaces or catheter materials (such as a polyethylene or poly propylene catheter material and/or surface).

The calixarene may be bonded to the surface of the intermittent catheter via radical grafting, preferably to a polyolefin surface or catheter material (such as a polyethylene or poly propylene catheter material and/or surface).

In preferred embodiments, the calixarene is bonded to an outer surface of the intermittent catheter. This allows for a high lubricity, non-stick, bacteria repellent outer coating, making the intermittent catheter easier to insert and remove and allowing it bacteria repellent properties that make it safer to use for a user.

In other preferred embodiments, the calixarene is bonded to both outer and inner surfaces of the intermittent catheter. This allows for both outer and inner surfaces to gain bacteria repellent properties from the coating calixarene, making the intermittent catheter safer to use for a user.

In some embodiments, the calixarene is coated over at least 50, 55, 60, 65, 70, 75, 80, 85, 90, 95, 96, 97, 98 or at least 99% of the outer surface area of the catheter, preferably at least 75% or at least 90% of the outer surface area of the catheter or between 75% and 100% of the outer surface.

In some embodiments, said outer surface of the intermittent catheter comprises a separate or further lubricating or bacteria-repellent agent coated on the surface, in addition to the calixarene.

In some embodiments, said further lubricating or bacteria-repellent agent is formed from a coating material selected from teh group comprising: silver-based, polytetrafluoroethylene, hydrogel, silicone, lecithin, salicylic acid, minocycline, rifampin, fluorinated ethylene propylene, polyvinylidone, polyvinyl compounds, polylactames, polyvinyl pyrrolidones, polysaccharides, heparin, dextran, xanthan gum, derivatised polysaccharides, hydroxy propyl cellulose, methyl cellulose, polyurethanes, polyacrylates, polyhydroxyacrylates, polymethacrylates, polyacrylamides, polyalkylene oxides, polyethylene oxides, polyvinyl alcohols, polyamides, polyacrylic acid, hydroxy ethylmethyl acrylate, polymethylvinyl ether, maleinic acid anyhydride, penicillin, neomycin sulfate, cephalothin, Bacitracin, phenoxymethyl penicillin, lincoymycin hydrochloride, sulfadiazine, methyl sulfadiazine, succinoylsulfathiazole, phthalylsulfathiazde, sulfacetamine, procaine penicillin, streptomycin, aureomycin, terramycin, terramycin, quaternary ammonium halides, cetyl pyridinium chloride, triethyl dodecyl ammonium bromide, hexachlorophene and nitrofurazone, or any combination thereof.

According to a second aspect of the invention, there is provided a method of manufacturing an intermittent catheter comprising providing an intermittent catheter, and bonding a calixarene onto a surface of the catheter.

The method may comprise manufacturing an intermittent catheter as described hereinabove for the first aspect of the invention. In preferred embodiments, the intermittent catheter is formed of a material of the group comprising: polyolefins, polyesters, polyacrylates, polyamides, thermoplastic elastomeric material, polyether block amide, thermoplastic vulcanizates, thermoplastic copolyesters, thermoplastic polyamides, fluororubber, and water disintegrable or enzymatically hydrolysable material or combinations, blends or co-polymers of any of the above materials.

The method may comprise bonding the calixarene to the surface of the intermittent catheter via covalent bonds, ionic bonds, hydrogen bonds, or Van der Waals forces.

According to a third aspect of the invention there is provided use of a calixarene as a bacteria-repellent agent on a surface of an intermittent catheter. In some embodiments the calixarene is used as a coating material, coated over at least 75% of the surface, preferably outer surface, of the intermittent catheter. In some embodiments, the calixarene is coated over between 75% and 100% of the outer surface of the intermittent catheter.

According to a fourth aspect of the invention there is provided use of a calixarene as a lubricating agent on a surface of an intermittent catheter. In some embodiments the calixarene is used as a coating material, coated over at least 75% of the surface, preferably outer surface, of the intermittent catheter. In some embodiments, the calixarene is coated over between 75% and 100% of the outer surface of the intermittent catheter.

### DETAILED DESCRIPTION OF THE INVENTION

In order that the invention may be more clearly understood embodiments thereof will now be described, by way of example only:

### Example 1

A first embodiment of an intermittent catheter of the invention comprises an intermittent catheter formed of a thermoplastic elastomeric material. The outer surface of the intermittent catheter is coated with a calixarene of formula:

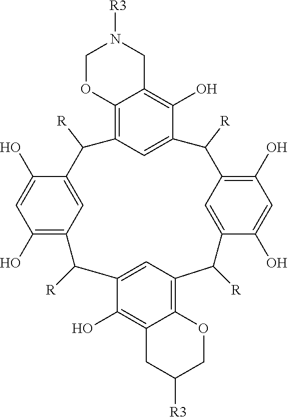

wherein R is —CH₂CH₂CH₂CH₂CH₂CH₂CH₂CH₂CH₂CH₂OC(O)O—(CH₂CH₂O)₇—CH₃, and R³ is -CH₂CH₂CH₂Si(OEt)₃.

The calixarene may be prepared as described in GB2498356.

The silane groups present in the calixarene act as surface-linker groups that allow the calixarene to bond to the outer surface of the intermittent catheter.

The intermittent catheter is used in the conventional manner.

The combination of the calixarene and the thermoplastic elastomeric material on the outer surface of the intermittent catheter allows the intermittent catheter to possess high lubricity and bacteria repellent properties, making it both easier to insert and remove, and safer to use with a decreased risk of developing infections.

### Example 2

A second embodiment of an intermittent catheter of the invention comprises an intermittent catheter formed of polyethylene. The outer surface of the intermittent catheter is coated with a calixarene of formula (I); wherein X is H, Y is OH, Z is H, R is —CH₂C₈F₁₇, and n is 1.

The calixarene may be prepared as described in WO9739077.

The intermittent catheter is used in the conventional manner.

The combination of the calixarene and the polyethylene material on the outer surface of the intermittent catheter allows the intermittent catheter to possess high lubricity and bacteria repellent properties, making it both easier to insert and remove, and safer to use with a decreased risk of developing infections.

### Example 3

A third embodiment of the invention comprises an intermittent catheter that is formed of a thermoplastic elastomeric material (polyolefin—either polyethylene or polypropylene). The outer surface of the intermittent catheter is coated with a silver alloy and hydrogel coating as a further bacteria-repellent coating material, alongside a calixarene of formula

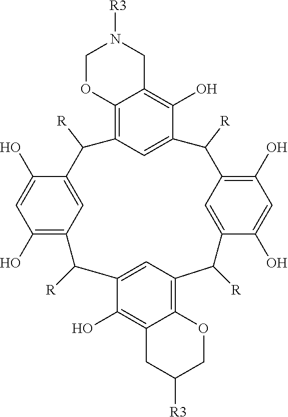

wherein R is —CH₂CH₂CH₂CH₂CH₂CH₂CH₂CH₂CH₂CH₂OC(O)O—(CH₂CH₂O)₇—CH₃, and R³ is —CH₂CH₂CH₂Si(OEt)₃.

The calixarene may be prepared as described in GB2498356.

The present intermittent catheter is used in the conventional manner.

As in Example 1, the combination of the calixarene and the thermoplastic elastomeric material on the surface of the intermittent catheter allows the intermittent catheter to possess high lubricity and bacteria repellent properties, making it both easier to insert and remove, and safer to use, with a decreased risk of developing infections. The additional silver alloy and hydrogel coating further inhibits bacterial growth on the intermittent catheter surface and enhances its lubricity further, making it easier and less irritating to insert and remove the intermittent catheter.

Suitable methods for coating the intermittent catheters of Examples 1-3 with the respective calixarenes are known to those skilled in the art. They include methods described in the following: WO97/39077, WO2005/112570, U.S. Pat. Nos. 6,702,850, 6,602,287, 5,053,048, 7,070,798 and US 2002/0102405.

The above embodiments are described by way of example only. Many variations are possible without departing from the scope of the invention as defined in the appended claims.

1. An intermittent catheter comprising a surface comprising a calixarene.

2. An intermittent catheter as claimed in claim 1, wherein said calixarene is bonded to the surface of the intermittent catheter via one or more surface-linker groups on a rim of the calixarene, said surface-linker groups being bonded to the surface of the device via covalent bonds, ionic bonds, hydrogen bonds, or Van der Waals forces.

3. An intermittent catheter according to claim 2, wherein an opposing rim of the calixarene is substituted by one or more polyethylene glycol, polypropylene glycol or polytrimethylene glycol groups, or a mixture thereof, forming glycol surface-linker groups.

4. An intermittent catheter according to claim 3, wherein said glycol linker groups are attached to the calixarene via (C₃ to C₁₆)alkyl spacer groups, said glycol groups, each independently, have from 2 to 250 repeating glycol units and may be optionally terminated by hydrogen or (C₁ to C₄)alkyl, said (C₃ to C₁₆)alkyl spacer groups may be optionally substituted by one or more fluoro, methyl or ethyl groups and may optionally contain one or more unsaturated bonds.

5. An intermittent catheter according to claim 4, wherein said glycol groups are attached to the alkylene spacer group directly via the oxygen of the glycol or via another linker group.

6. An intermittent catheter according to claim 3, wherein the glycol linker group is selected from carbonate, carbamate, urea, phosphate and triazole.

7. An intermittent catheter according to a claim 2, wherein the calixarene is bonded to the surface of the intermittent catheter via 2 to 8 surface-linker groups.

8. An intermittent catheter according to claim 3, wherein a rim of the calixarene is substituted by one or more polyethylene glycol groups.

9. (canceled)

10. An intermittent catheter according to claim 1, wherein said calixarene is derived from a compound of formula (I) 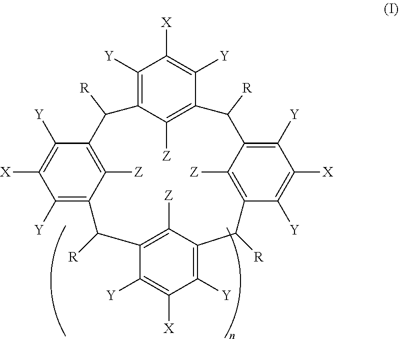
wherein
X is H, (C₁-C₃₀)alkyl, NH₂, NH(C₁-C₃₀)alkyl, N(C₁-C₃₀)alkyl2, CH₂NH(C₁-C₃₀)alkyl, OH, O(C₁-C₃₀)alkyl or OCH₂CO₂(C₁-C₃₀)alkyl;
Y is OH, O(C₁-C₃₀)alkyl, OCH₂CO₂(C₁-C₃₀)alkyl, H, (C₁-C₃₀)alkyl, NH₂, NH(C₁-C₃₀)alkyl, N(C₁-C₃₀)alkyl₂ or CH₂NH(C₁-C₃₀)alkyl;
Z is H, OH or methyl;
n is 1, 3 or 5; and
R is —(C₁-C₃₀)alkyl- or —(C₁-C₃₀)alkylene-L¹-G-R¹ wherein said alkyl or alkylene may be optionally substituted by one or more fluoro, methyl or ethyl groups and may optionally contain one or more unsaturated bonds;
L¹ is a bond or a linking group;
G is —O(CH₂CH₂O)ₘ-, —O(CH₂CH(CH₃)O)ₘ-, —(O(CH(CH₃)CH₂O)ₘ-, or —O(CH₂CH₂CH₂O)ₘ-;
m is 2 to 250;
R¹ is H or (C₁-C₃₀)alkyl;
and wherein each X, Y, Z, R and R¹ group may be the same or different.

11. An intermittent catheter according to claim 10, wherein X is H and Y is OH.

12. An intermittent catheter according to any one of claim 10, wherein X is H and R is —CH₂C₈F₁₇.

13. An intermittent catheter according to claim 10, wherein R is —(C₁₀)alkylene-L¹-G-R¹.

14. An intermittent catheter according to claim 10, wherein L¹ is a linking group selected from carbonate, carbamate, urea, phosphate and triazole.

15. An intermittent catheter according to claim 10, wherein G is —O(CH₂CH₂O)ₘ-.

16. An intermittent catheter according to claim 10, wherein R¹ is H or methyl.

17. An intermittent catheter according to claim 10, wherein m is 3 to 150.

18. An intermittent catheter according to claim 10, wherein said calixarene is bonded to the surface of the device via surface-linker groups substituted for any one or more of the X or Y substituents, or a combination thereof

19. An intermittent catheter according to claim 18, wherein the surface-linker groups X or Y, or a combination thereof, on the calixarene are derived from acid chloride, chloroformate, vinyl, acrylate, or silane functional groups.

20. (canceled)

21. An intermittent catheter according to claim 10, wherein R is —(C₃-C₁₆)alkylene-OH or —(C₂-C₁₅)alkylene-CH₂═CH₂ wherein said alkylene may be optionally substituted by one or more fluoro, methyl or ethyl groups and may optionally contain one or more unsaturated bonds;
and wherein each X, Y, Z and R group may be the same or different;
with the proviso that when X is H, OH, CH₃ or OCH₃; Y is OH; Z is H; and n is 1; R is not —(C₈)alkylene-CH₂═CH₂.

22. An intermittent catheter according to claim 18, wherein said calixarene is 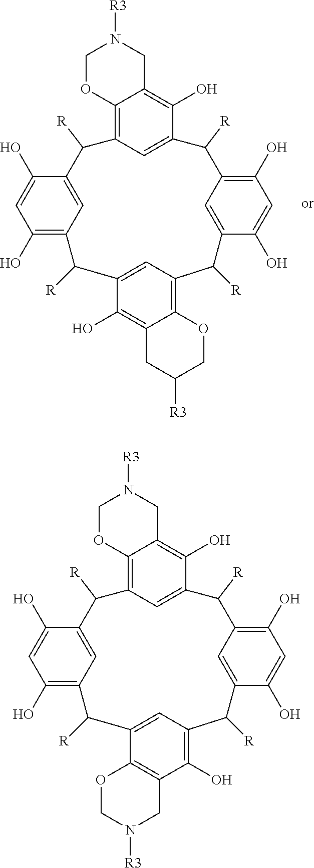
wherein
R is —(C₁₀)alkylene-OC(O)O—(CH₂CH₂O)ₘ—OH; and
R³ is (C₃)alkylene-Si(OEt)₃ or a combination thereof.

23-27. (canceled)
